(19) United States
(12) Patent Application Publication
(10) Pub. No.: US 2024/0153048 A1
(43) Pub. Date: May 9, 2024

(54) ARTIFACT REMOVAL IN ULTRASOUND IMAGES

(71) Applicant: GE Precision Healthcare LLC, Wauwatosa, WI (US)

(72) Inventors: Pavan Annangi, Bengaluru (IN); Anders R. Sørnes, Oslo (NO); Prasad Sudhakara Murthy, Bengaluru (IN); Bhushan D. Patil, Bengaluru (IN); Erik Normann Steen, Horten (NO); Tore Bjaastad, Molde (NO); Rohan Keshav Patil, Bengaluru (IN)

(21) Appl. No.: 18/052,820

(22) Filed: Nov. 4, 2022

(51) Int. Cl. G06T 5/00; G06T 5/10

(52) U.S. Cl. CPC G06T 5/006; G06T 5/10; G06T 2207/10132; G06T 2207/20024; G06T 2207/20048

(57) ABSTRACT

Methods and systems are provided for removing visual artifacts from a medical image acquired during a scan of an object, such as a patient. In one example, a method for an image processing system comprises receiving a medical image; performing a wavelet decomposition on image data of the medical image; performing one or more 2-D Fourier transforms on wavelet coefficients resulting from the wavelet decomposition; removing image artifacts from the Fourier coefficients determined from the 2-D Fourier transforms using a filter; reconstructing the medical image using the filtered Fourier coefficients; and displaying the reconstructed medical image on a display device of the image processing system.

### TECHNICAL FIELD

Embodiments of the subject matter disclosed herein relate to ultrasound imaging, and more particularly, to improving image quality for ultrasound imaging.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. For example, a medical imaging device such as an ultrasound imaging device may be used to obtain images of a heart, uterus, liver, lungs, and various other anatomical regions of a patient. Ultrasound images of the internal structures may be saved for later analysis by a clinician to aid in diagnosis and/or displayed on a display device in real time or near real time.

The quality of ultrasound acquisitions depends on several factors, including beamspacing, number of transmits, a size of transmit and receive apertures, a reconstruction method, a number of overlapping receive lines used for coherent or incoherent reconstruction, etc. For acquisition of ultrasound images at high frame rates or volume rates, there may be a trade-off between the number of transmits and a size of an aperture that can be used. Acquisitions with a reduced number of transmits may generate artifacts that look like streaks or stripes. The artifacts may be more pronounced with volume probes that use extremely sparse transmits to achieve high volume rates.

Several approaches can be taken to remove the artifacts. One method is a model-based signal loss compensation. However, while such a solution may work for small transmit spacing values, it may not work for larger transmit spacing values. Moreover, it would not be robust with respect to variable subjects and scanning situations, and it would rely on an accurate model of the imaging system. U.S. Pat. No. 5,987,347 discloses a method for eliminating streaks from a medical image by replacing pixels at streak locations with pixels from a filtered version of the medical image. However, methods such as these may rely on the streaks having well defined boundaries, which may not be the case (for example, with ultrasound images). Additionally, the methods may not work when a width of the streaks are greater than a small number of pixels (e.g., one pixel).

### SUMMARY

In one embodiment, a method for an image processing system comprises receiving a medical image; performing a wavelet decomposition on image data of the medical image to generate a set of wavelet coefficients; identifying a first portion of the wavelet coefficients including image artifact data, and a second portion of the wavelet coefficients not including the image artifact data; performing one or more 2-D Fourier transforms on the first portion of the wavelet coefficients to generate Fourier coefficients, the Fourier coefficients including the image artifact data; removing the image artifact data from the Fourier coefficients generated from the 2-D Fourier transforms, using a filter; performing an inverse 2-D Fourier transform on the filtered Fourier coefficients to generate updated wavelet coefficients corresponding to the first portion; reconstructing an artifact-removed image from the updated wavelet coefficients corresponding to the first portion of the wavelet coefficients and the second portion of the wavelet coefficients; and displaying the reconstructed artifact-removed image on a display device of the image processing system. For example, the medical image may be an ultrasound image, where stripe artifacts are removed from the ultrasound image by applying the 2-D Fourier transforms after wavelet decomposition and filtering with a notch filter.

By filtering image data processed via both the wavelet decomposition and the 2-D Fourier transforms, artifacts such as stripes may be removed even when stripe boundaries are not well-defined, and stripes of a range of widths and spatial frequency may be removed from the image data. (It should be appreciated that removing artifacts, artifact removal, and/or artifact-removed images, as described herein, refer to a process by which artifacts in an image may be substantially reduced. Total elimination of artifacts using the artifact removal process described herein may not be possible, and in some cases, traces of the artifacts may remain in the image after the artifact removal process has been applied.) Additionally, an acquisition of medical images with less or no artifacts may be performed at a higher volume rate than an acquisition using different artifact-removal techniques, with almost no changes to a workflow of an operator of the image processing system. Further, smaller aperture acquisitions typically produce artifact-free images at a lower resolution, while larger aperture acquisitions produce higher resolution images, though with artifacts. In contrast, the method disclosed herein may allow an imaging system to utilize larger transmit apertures, thereby generating higher quality images, without showing artifacts. An additional advantage of the solution provided herein is that the method may work across various different implementations and settings, by modifying a design of the wavelet type, the notch filter, and/or a combined use of both. The different implementations and settings may include, for example, different decimation factors, different aperture settings, both 2-D and 4-D ultrasound probes, over different reconstruction planes (azimuth/elevation) and with different reconstruction methods (e.g., Retrospective Transmit Beamforming (RTB), Synthetic Transmit Beamforming (STB), incoherent STB, etc.).

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of an ultrasound system;

FIG. 2 is a schematic diagram illustrating a system for generating ultrasound images, according to an exemplary embodiment;

FIG. 3A is an image of a narrowly focused ultrasound beam, as prior art;

FIG. 3B is a first ultrasound image generated using a narrowly focused ultrasound beam, as prior art;

FIG. 3C is a second ultrasound image generated using a narrowly focused ultrasound beam, as prior art;

FIG. 3D is an image of a less focused ultrasound beam, as prior art;

FIG. 3E is a third ultrasound image generated using a less focused ultrasound beam, as prior art;

FIG. 3F is a fourth ultrasound image generated using a less focused ultrasound beam, as prior art;

FIG. 4 is a flow chart illustrating an example method for removing artifacts from an ultrasound image, according to an embodiment;

FIG. 5A is an ultrasound image inputted into a wavelet transform process, according to an embodiment;

FIG. 5B is a composite image representing a wavelet transform process applied to an ultrasound image, according to an embodiment;

FIG. 5C is an image representing a result of a Fourier transform process applied to an ultrasound image, according to an embodiment;

FIG. 5D is an image representing a notch filter applied to a result of a Fourier transform process applied to an ultrasound image, according to an embodiment;

FIG. 5E is an image resulting from applying a notch filter to a transformed ultrasound image, according to an embodiment; and

FIG. 5F is a reconstructed image with reduced artifacts, according to an embodiment.

### DETAILED DESCRIPTION

Medical ultrasound imaging typically includes the placement of an ultrasound probe including one or more transducer elements onto an imaging subject, such as a patient, at the location of a target anatomical feature (e.g., abdomen, chest, etc.). Images are acquired by the ultrasound probe and are displayed on a display device in real time or near real time (e.g., the images are displayed once the images are generated and without intentional delay). The operator of the ultrasound probe may view the images and adjust various acquisition parameters and/or the position of the ultrasound probe in order to obtain high-quality images of the target anatomical feature (e.g., the heart, the liver, the kidney, or another anatomical feature). The acquisition parameters that may be adjusted include transmit frequency, transmit depth, gain (e.g., overall gain and/or time gain compensation), beamspacing, cross-beam, beam steering angle, beamforming strategy, frame averaging, size of transmit and receive apertures, reconstruction method, number of overlapping receive lines used for coherent/incoherent reconstruction, and/or other parameters.

Varying the acquisition parameters to acquire an optimal image (e.g., of a desired quality) can be challenging, and may entail tradeoffs between different acquisition parameters. In particular, for 2-D acquisitions relying on high frame rates, or 3-D acquisitions relying on high volume rates, a tradeoff may exist between a number of transmits (e.g., an individual transmission of an ultrasound beam) and a size of a transmit aperture that may be used. For example, a typical frame rate for 2-D acquisitions may be 50 frames per second, and a typical frame rate for 3-D acquisitions may be at an equivalent of 320 individual planes per second. In other words, for 3-D acquisitions, to cover an entire volume at a volume rate of 20-50 fps, the individual planes comprising the 3-D volume will be acquired at 320 planes per second, such that the 2-D or “plane” image quality would be comparable to what one would get at 320 fps in regular 2-D.

An ultrasound beam may be focused, using a lens such as a concave crystal lens or an acoustic lens, to generate a focal zone of a desired length and position with respect to the transmit aperture. The focal zone is an area within which an ultrasound beam is in high focus, centered around a focal point at which the ultrasound beam is in a highest focus. When performing a scan, the ultrasound beam may be focused such that a depth of a portion of a scanned object that is desired to be imaged (e.g, an anatomical region of interest (ROI)) is within the focal zone. As the size of the transmit aperture is increased, a depth of the focal zone may change, and a width of the ultrasound beam within the focal zone may be reduced.

Different beamforming techniques may be used to synthetically modify a transmit beam used by ultrasound systems to acquire ultrasound data that is used to generate images. As one example, RTB is used to form a synthetically focused ultrasound image using standard, scanned, and focused or defocused ultrasound transmissions. More particularly, RTB is a synthetic focus technique that uses standard, scanned-beam transmit data, dynamic receive focusing, and coherent combination of time-aligned data from multiple transmits to form images. As a second example, STB can be used to generate images, by combining co-linear receive lines from successive partially overlapping transmits incoherently or coherently.

For each transmit, an amount of time taken for the ultrasound beam to be transmitted to a receiver and for a reflection of the beam to be reflected back will be fixed in a way depending on physical properties of a scanned object. Thus, an amount of time spent in generating an image may increase in proportion to an increase in the number of transmits. To generate an image of a desired quality within a desired amount of time may not be easily achievable, since the number of transmits available will be constrained by the desired amount of time.

To generate an image that sufficiently covers an anatomical region of interest (ROI), the size of the aperture and/or the degree of focusing of the ultrasound beam may be reduced, resulting in a less focused ultrasound beam that images a wider portion of the ROI. Alternatively, the size of the aperture and/or the focusing of the ultrasound beam may be increased to generate a more narrowly focused ultrasound beam. When the ultrasound beam is more narrowly focused, the beamspacing of the ultrasound beams between transmits (e.g., a physical spacing between a first ultrasound beam of a first transmit and a second ultrasound beam of a second transmit) is decreased in order to maintain an overlap between the events. Increasing the focusing may generate a higher quality (e.g., higher resolution) image than using the less focused ultrasound beam. However, it may also result in artifacts, such as vertical stripes in resulting images. Decreasing the beamspacing may increase the quality of the images and reduce these artifacts, but may increase the number of transmits, thereby causing the time taken for each acquisition to exceed the desired amount of time for image acquisition. Thus, to address the issue of generating high quality images with a narrowly focused ultrasound beam without decreasing beamspacing, the inventors herein propose methods and systems for removing artifacts from images generated at the increased beamspacing.

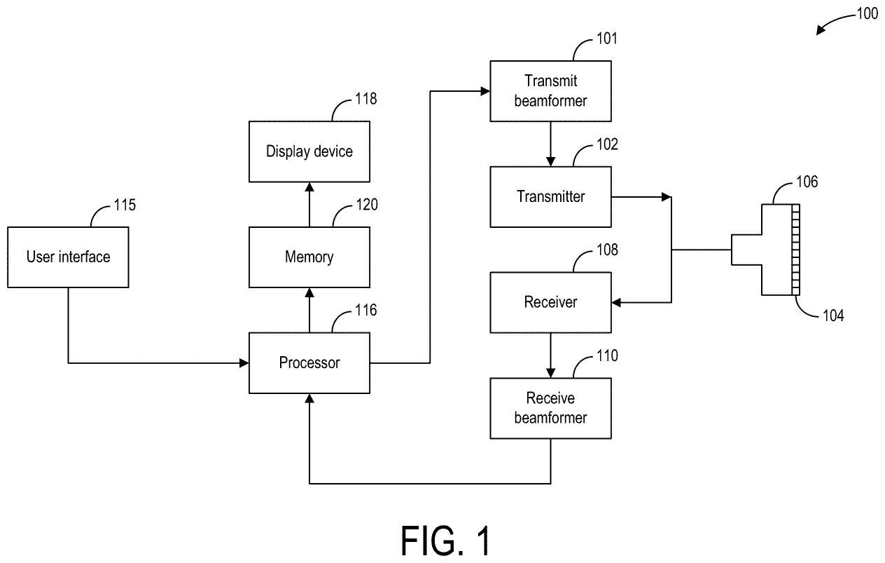

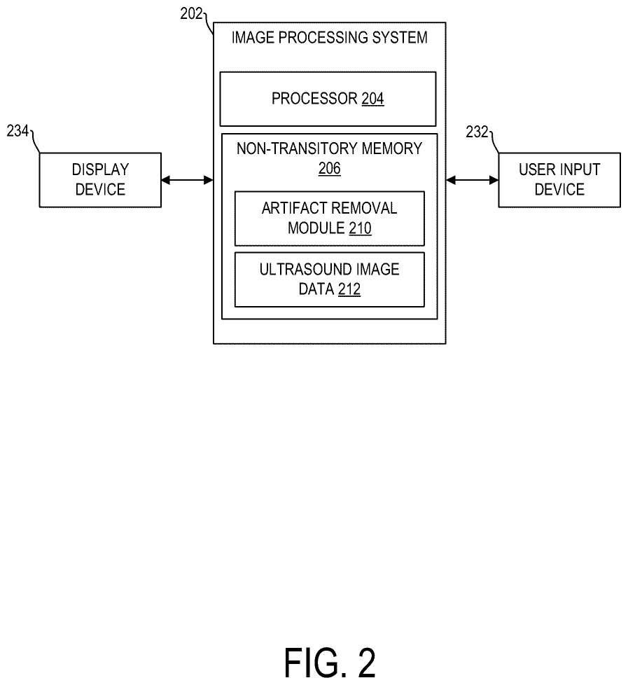

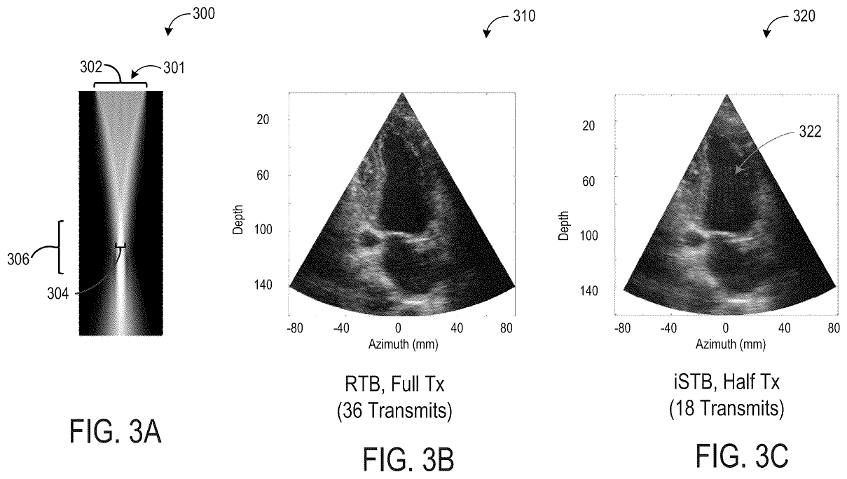

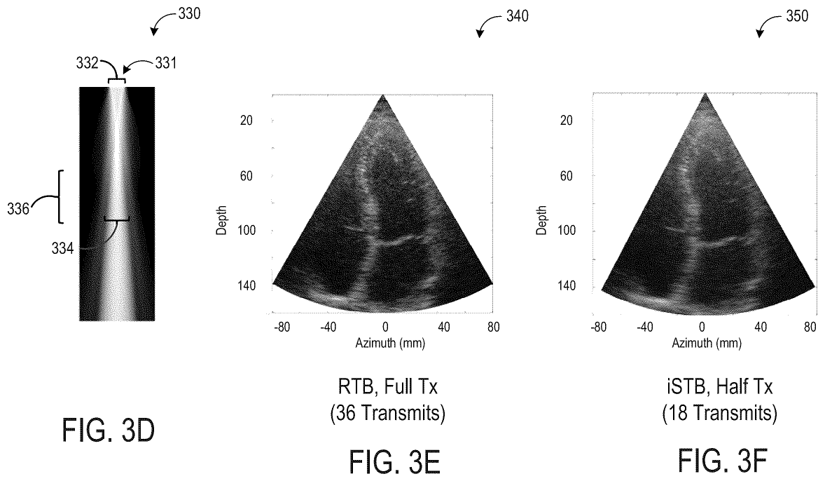

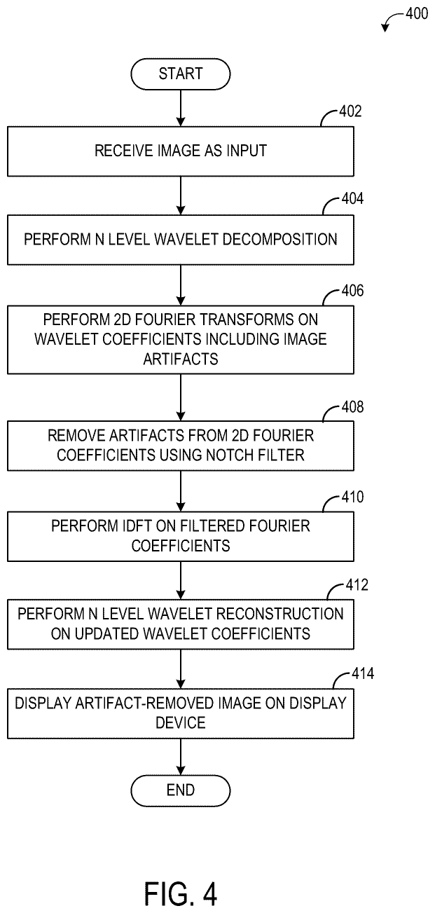

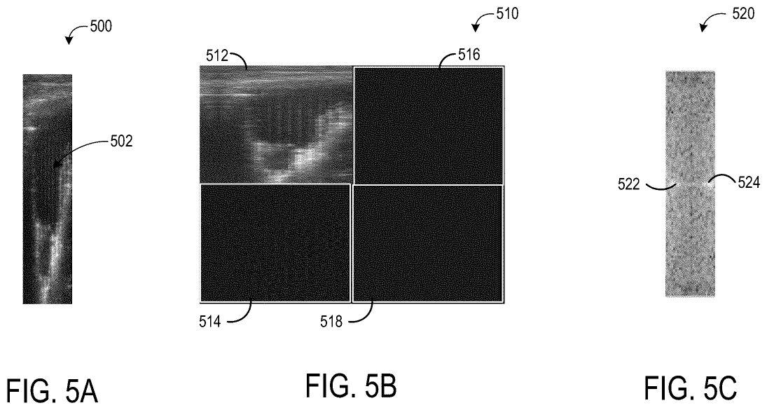

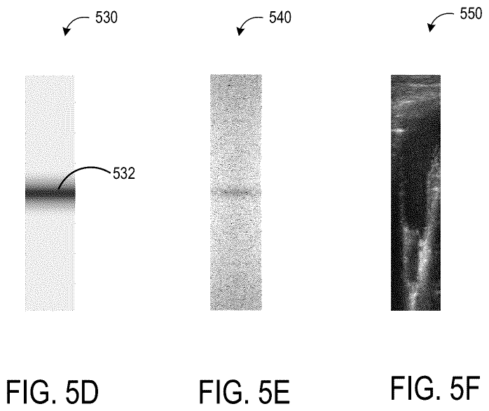

An example ultrasound system including an ultrasound probe, a display device, and an imaging processing system are shown in FIG. 1. Via the ultrasound probe, ultrasound images may be acquired and displayed on the display device. As described above, the images may be acquired using various acquisition scan parameters, such as transmit frequency and depth. An image processing system, as shown in FIG. 2, includes an artifact removal module in non-transitory memory, which may include code that when executed, removes artifacts from images generated by the image processing system. FIG. 3B shows an exemplary ultrasound image generated using a narrowly focused ultrasound beam, as shown in FIG. 3A. When a number of transmits is reduced by half, artifacts (e.g., vertical stripes) are generated, as shown in the ultrasound image of FIG. 3C. In comparison, when a less focused beam shown in FIGURE. 3-D is used, a quality of a resulting image may be lower than when the narrowly focused ultrasound beam is used, as shown in FIG. 3E. and the image quality may be further lowered when the number of transmits is reduced, as shown in FIG. 3F. To remove artifacts generated by increasing a beamspacing of a narrowly focused ultrasound beam, various transforms and filters may be applied to image data by following a procedure such as the method shown in FIG. 4. Visual depictions of various steps of the procedure are shown in FIGS. 5A-5F.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound system 100 may be used to train a machine learning model. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and/or a graphical user interface displayed on a display device 118.

The ultrasound imaging system 100 also includes a processor 116 to control the transmit beamformer 101, the transmitter 102, the receiver 108, and the receive beamformer 110. The processer 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2-D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by filtering the data to remove artifacts, as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2-D or 3-D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

Ultrasound images acquired by ultrasound imaging system 100 may be further processed. In some embodiments, ultrasound images produced by ultrasound imaging system 100 may be transmitted to an image processing system, such as the image processing system described below in reference to FIG. 2, where in some embodiments, the ultrasound images may be analyzed and/or edited as described herein. For example, an artifact removal procedure may be applied to image data to remove artifacts from the images prior to displaying the images on display device 118, as described below in reference to FIGS. 4 and 5A-5F.

Although described herein as separate systems, it will be appreciated that in some embodiments, ultrasound imaging system 100 includes an image processing system. In other embodiments, ultrasound imaging system 100 and the image processing system may comprise separate devices.

Referring to FIG. 2, an image processing system 202 is shown, in accordance with an embodiment. In some embodiments, image processing system 202 is incorporated into the ultrasound imaging system 100. For example, the image processing system 202 may be provided in the ultrasound imaging system 100 as the processor 116 and memory 120. In some embodiments, at least a portion of image processing 202 is disposed at a device (e.g., edge device, server, etc.) communicably coupled to the ultrasound imaging system via wired and/or wireless connections. In some embodiments, at least a portion of image processing system 202 is disposed at a separate device (e.g., a workstation) which can receive images from the ultrasound imaging system or from a storage device which stores the images/data generated by the ultrasound imaging system. Image processing system 202 may be operably/communicatively coupled to a user input device 232 and a display device 234. The user input device 232 may comprise the user interface 115 of the ultrasound imaging system 100, while the display device 234 may comprise the display device 118 of the ultrasound imaging system 100, at least in some examples.

Image processing system 202 includes a processor 204 configured to execute machine readable instructions stored in non-transitory memory 206. Processor 204 may be any suitable processor, processing unit, or microprocessor, for example. Processor 204 may be a multi-processor system, and, thus, may include one or more additional processors that are identical or similar to each other and that are communicatively coupled via an interconnection bus. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Non-transitory memory 206 may include one or more data storage structures, such as optical memory devices, magnetic memory devices, or solid-state memory devices, for storing programs and routines executed by processor(s) 106 to carry out various functionalities disclosed herein. Non-transitory memory 206 may include any desired type of volatile and/or non-volatile memory such as, for example, static random access memory (SRAM), dynamic random access memory (DRAM), flash memory, read-only memory (ROM), etc.

Non-transitory memory 206 may include an artifact removal module 210, which comprises instructions for removing artifacts from ultrasound images. In various embodiments, the ultrasound images may be generated by an ultrasound system such as ultrasound imaging system 100 of FIG. 1. In some embodiments, artifact removal module 210 is not disposed at image processing system 202.

Non-transitory memory 206 may further store ultrasound image data 212, such as ultrasound images captured by the ultrasound imaging system 100 of FIG. 1. The ultrasound images of the ultrasound image data 212 may comprise ultrasound images that have been acquired by the ultrasound imaging system 100 at different scan settings, such as different transmit frequencies, different beamspacing, different gains, different depths, different TGCs, different transmit/receive aperture sizes, different frame averaging, etc.

In some embodiments, the non-transitory memory 206 may include components disposed at two or more devices, which may be remotely located and/or configured for coordinated processing. In some embodiments, one or more aspects of the non-transitory memory 206 may include remotely-accessible networked storage devices configured in a cloud computing configuration.

User input device 232 may comprise one or more of a touchscreen, a keyboard, a mouse, a trackpad, a motion sensing camera, or other device configured to enable a user to interact with and manipulate data within image processing system 202. In one example, user input device 232 may enable a user to make a selection of an ultrasound image to use in training a machine learning model, to indicate or label a position of an interventional device in the ultrasound image data 212, or for further processing using a trained machine learning model.

Display device 234 may include one or more display devices utilizing virtually any type of technology. In some embodiments, display device 234 may comprise a computer monitor, and may display ultrasound images. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view ultrasound images produced by an ultrasound imaging system, and/or interact with various data stored in non-transitory memory 206.

It should be understood that image processing system 202 shown in FIG. 2 is for illustration, not for limitation. Another appropriate image processing system may include more, fewer, or different components.

Referring now to FIGS. 3A-3C, an example of artifact generation due to reducing a number of transmits when using a narrowly focused ultrasound beam is shown.

FIG. 3A is an image of a narrowly focused ultrasound beam 300. Narrowly focused ultrasound beam 300 may be generated by using a first aperture 301 of a first, large size, with a first transmit aperture width 302. Narrowly focused ultrasound beam 300 may be focused using a plurality of individually delayed pulses from the collection of small transducer elements comprising the aperture. As a result of using first transmit aperture 301 and focusing the ultrasound beam via the lens, narrowly focused ultrasound beam 300 may be focused to a first target width 304 at a focal zone 306, where the first target width 304 is smaller than first aperture width 302.

FIG. 3B shows a first ultrasound image 310 generated using retrospective transmit beamforming (RTB), where a first number of transmits are performed with a narrowly focused ultrasound beam, such as the narrowly focused ultrasound beam 300 of FIG. 3A. The first number of transmits may be a number of transmits that completely images a scanned area of a subject of an ultrasound scan, where a beamspacing of the transmits is sufficiently dense to generate an image of the scanned area without any gaps between transmits. For example, the first number of transmits may be 36. As a result of using the narrowly focused ultrasound beam with the sufficiently narrow beamspacing, first ultrasound image 310 may represent a high-quality image of the scanned area.

FIG. 3C shows a second ultrasound image 320 of the same scanned area as FIG. 3B generated using incoherent synthetic transmit beamforming (iSTB), where a second number of transmits are performed with a narrowly focused ultrasound beam, such as the narrowly focused ultrasound beam 300 of FIG. 3A. The second number of transmits may be a smaller number of transmits than the first number of transmits of FIG. 3B. For example, the second number of transmits generated using iSTB may be 18 (e.g., half of the number of transmits of FIG. 3B, using RTB). To generate an image that covers the scanned area with half the number of transmits, a wider beamspacing (e.g., wider than the beamspacing of FIG. 3B) may be used between transmits. A result of using half the number of transmits is that a quality of second ultrasound image 320 may be decreased with respect to the quality of first ultrasound image 310. For example, a spatial resolution of second ultrasound image 320 may be lower than first ultrasound image 310. Additionally, as a result of the smaller number of transmits and the wider beamspacing, gaps between transmits may cause one or more artifacts 322, which appear in FIG. 3C as vertical stripes.

In contrast to FIGS. 3A-3C, FIGS. 3D-3F show exemplary images reconstructed from image data acquired using a less focused ultrasound beam. By using a less focused ultrasound beam, image data is acquired over a larger (e.g., wider) portion of the scanned area during each transmit.

FIG. 3D is an image of a less focused ultrasound beam 330. Less focused ultrasound beam 330 may be generated by using a second aperture 331, where second aperture 331 is smaller than first aperture 301 of FIG. 3A. Second aperture 331 has a second aperture width 332, which is smaller than first aperture width 302 of FIG. 3A. As a result of using the second, smaller aperture 331, less focused ultrasound beam 330 may be focused to a second target width 334 at a focal zone 336, where second target width 334 is greater than second aperture width 332, and greater than first target width 304 of FIG. 3A.

FIG. 3E shows a third ultrasound image 340 of a similar scanned area as FIGS. 3B and 3C generated using RTB, where a third number of transmits are performed with a less focused ultrasound beam, such as the less focused ultrasound beam 330 of FIG. 3C. The third number of transmits may be the same as the first number of transmits of FIG. 3B (e.g., 36), which completely image the scanned area of the subject. In other words, the beamspacing may be sufficiently dense (e.g., 0) that there are no gaps between the less focused ultrasound beams of each transit. In fact, the beamspacing may now be excessively dense when compared to the beam, establishing an unnecessarily large overlap between the transmit beams. However, as a result of using the less focused ultrasound beam, third ultrasound image 340 may have a lower spatial resolution than first ultrasound image 310, whereby third ultrasound image 340 may represent a lower quality image than first ultrasound image 310.

FIG. 3F shows a fourth ultrasound image 350 generated using iSTB, where a fourth number of transmits are performed with the less focused ultrasound beam 330 of FIG. 3D. The fourth number of transmits may be a smaller number of transmits than the third number of transmits of FIG. 3E. For example, the fourth number of transmits may be half of the number of transmits of FIG. 3E (e.g., 18 transmits). To generate an image that covers the scanned area with only half the number of transmits, a wider beamspacing (e.g., wider than the beamspacing of FIG. 3E) may be used between transmits. As a result of the smaller number of transmits and the wider beamspacing, fourth ultrasound image 350 may be of a lower quality than third ultrasound image 340 of FIG. 3E, but the reduction in quality may not be as large as between 310 and 320, since the wider transmit beam allows larger transmit beam spacing. However, as a result of the ultrasound beam being less focused than in second ultrasound image 320 of FIG. 3C (e.g., as a result of width 334 of FIG. 3D being greater than width 304 of FIG. 3A), no artifacts may be caused in fourth ultrasound image 350 due to gaps between transmits.

Turning now to FIG. 4, a method 400 is shown for removing visual artifacts from an ultrasound image, such as the vertical stripes seen in FIG. 3C. As described above, the vertical stripes may appear when the transmit beams spacing is similar or larger than the transmit beam width. It should be appreciated that while method 400 describes removing vertical stripe artifacts from an ultrasound image, one or more steps of method 400 may also be used to remove stripe artifacts of different orientations, or other types of visual artifacts, from the ultrasound image. It should be appreciated that while FIG. 4 is described in reference to an ultrasound image (e.g., B-mode, Color Doppler, M-mode, Color M-mode, spectral Doppler, Elastography, TVI, strain, strain rate, etc.), one or more steps of FIG. 4 may be applied to other types of medical images without departing from the scope of this disclosure.

Method 400 may be carried out according to instructions stored in non-transitory memory of a computing device, such as image processing system 202 of FIG. 2. Method 400 may be carried out between a first time at which the ultrasound image is generated, and a second time at which the ultrasound image is displayed on a display device, such as display device 234 of image processing system 202 and/or display device 118 of ultrasound imaging system 100 of FIG. 1. For example, in various embodiments, ultrasound images may be processed via method 400 “on-the-fly” and displayed in real time with the visual artifacts removed as an ultrasound procedure is being performed. In other embodiments, ultrasound images may be generated from a scan performed at a first time. The ultrasound images may be stored in the image processing system until a second time, at which a radiologist wishes to view the ultrasound images. At the second time, the radiologist may display the ultrasound images on a display device of the image processing system. For example, the radiologist may wish to view ultrasound images acquired from a patient in the past. Prior to displaying the ultrasound images on the display device, method 400 may be applied to remove artifacts from the ultrasound images.

Method 400 begins at 402, where method 400 includes receiving an ultrasound image as input. The ultrasound image may be generated from raw data acquired by an ultrasound probe, such as ultrasound probe 106 of imaging system 100 of FIG. 1, and reconstructed using RTB, STB, or a different beamforming/image reconstruction method. In some embodiments, the ultrasound image may be received from ultrasound image data stored in an image processing system (e.g., ultrasound image data 212 of image processing system 202). For example, the ultrasound image may be a B-mode ultrasound image, or a Doppler ultrasound image, or a different type of ultrasound image.

At 404, method 400 includes performing a wavelet decomposition at N levels to generate a set of wavelet coefficients, where each level N corresponds to a different scale of the ultrasound image. For example, a first wavelet decomposition may be performed at a first scale; a second wavelet decomposition may be performed at a second scale; a third wavelet decomposition may be performed at a third scale; and so on. At each scale/level of the wavelet decomposition, the wavelet decompositions may extract artifacts corresponding to the scale. In other words, the first wavelet decomposition may extract a first set of artifacts at a first scale; the second wavelet decomposition may extract a second set of artifacts at a second scale; the third wavelet decomposition may extract a third set of artifacts at a third scale; and so on.

Each wavelet decomposition may be a wavelet transform that indicates a correlation or similarity between a first, wavelet basis function and a second function of image data, where the image data may include artifacts. The wavelet basis function may be different for decompositions at different scales. For example, one wavelet basis function may be used at a first level N; a different wavelet basis function may be used at a second level N; a different wavelet basis function may be used at a third level N; and so on. An initial wavelet basis function may be selected for the decomposition at any level N. However, once the initial wavelet basis function is chosen, the wavelet basis functions for other levels become fixed, as they have exact relationships with each other. Thus, the selection of the initial wavelet basis function may depend on the nature of an artifact to be processed, and may include a process of trial and error.

In various embodiments, the wavelet transform may be a convolution of the first function with the second function, where a scalar product of the first function and the second function (a coefficient) is generated. In other embodiments, rather than a convolution, a lifting scheme may be implemented, or a different numerical scheme for performing a wavelet decomposition may be used. A high coefficient generated by the wavelet decomposition process may indicate a high degree of similarity between the first function and the second function, and a low coefficient may indicate a high degree of similarity between the first function and the second function. The coefficient represents artifact data at a relevant scale/level. Thus, N wavelet decompositions may be performed to generate N coefficients at N different scales/levels of the ultrasound image. The coefficients may be used to extract artifacts occurring at the different scales/levels.

The coefficients may also be used to extract artifacts occurring at different orientations in the ultrasound image. At each level of decomposition of an image, the wavelet decompositions involve three orientations: horizontal, vertical, and diagonal. For example, a first set of wavelets and decompositions may be applied to the ultrasound image to detect artifacts at a first orientation (e.g., vertical); a second set of wavelets and decompositions may be applied to the ultrasound image to detect artifacts at a second orientation (e.g., horizontal); a third set of wavelets and decompositions may be applied to the ultrasound image to detect artifacts at a third orientation (e.g., diagonal); and so on. It should be appreciated that the artefacts in the vertical direction are actually detected by a decomposition component with horizontal orientation, and vice versa.

Referring to FIG. 5A, an input ultrasound image 500 is shown, which includes a plurality of vertical stripes 502. Ultrasound image 500 may be reconstructed using one of various ultrasound beamforming/image reconstruction methods from raw signal acquisitions (e.g., RTB, STB, iSTB, etc.), as described above. When the wavelet decomposition is performed, four images are generated from input image 500, as shown in FIG. 5B.

FIG. 5B shows a composite image 510 including four images resulting from a wavelet decomposition process applied to input image 500. Composite image 510 includes an approximation image 512 of a first level wavelet decomposition of ultrasound image 500. Approximation image 512 includes a low frequency version of the initial input image, with the high frequencies available in the details. The artifacts typically belong to high frequencies, but they may have some overlap with low frequencies as well, and therefore approximation image 512 may still have artifacts. During the wavelet decomposition process, wavelet decomposition at one level of the N levels may be based on the approximation image 512 of the wavelet decomposition at a preceding level, rather than the input image 500. As approximation image 512 may still include artifacts, it may need to be further broken down into low and high frequencies in a subsequent iteration to tease out and filter the artifacts.

Composite image 510 includes a vertical artifact detail image 514 showing vertical artifact data (e.g., wavelet transform coefficients) extracted from ultrasound image 500. The vertical artifact data may correspond, for example, to the vertical stripes 502 (and shown in artifacts 322 of second ultrasound image 320 of FIG. 3C). The wavelet decomposition process may also extract horizontal artifact data from ultrasound image 500, shown in a horizontal artifact detail image 516, and diagonal artifact data from ultrasound image 500, shown in a diagonal artifact detail image 518. In FIG. 5B, vertical artifact detail image 514 includes a greater amount of artifact data than is seen in horizontal artifact detail image 516 and diagonal artifact detail image 518. In other words, ultrasound image 500 includes vertical stripes 502, and does not include visible horizontal or diagonal stripe artifacts. In other embodiments, the wavelet decomposition process may extract different types of artifacts having the same or different orientations.

Returning to FIG. 4, at 406, method 400 includes performing 2-D Fourier transforms on a portion of the wavelet coefficients generated during the wavelet decomposition process described above, where the portion of the wavelet coefficients includes artifact data. Thus, the 2-D Fourier transforms may be performed on some wavelet coefficients, and not on other wavelet coefficients. As described above, vertical artifacts are detected in horizontally oriented wavelet decompositions, and horizontal artifacts are detected in vertically oriented wavelet decompositions. For example, if vertical artifacts are seen in the ultrasound image, the 2-D Fourier transforms may be performed on a first portion of wavelet coefficients generated from horizontally oriented wavelet decompositions, and the 2-D Fourier transforms may not be performed on a second portion of wavelet coefficients generated from vertically or diagonally oriented wavelet decompositions. Alternatively, if horizontal artifacts are seen in the ultrasound image, the 2-D Fourier transforms may be performed on a first portion of wavelet coefficients generated from vertically oriented wavelet decompositions, and the 2-D Fourier transforms may not be performed on a second portion of wavelet coefficients generated from horizontally or diagonally oriented wavelet decompositions.

Referring briefly to FIG. 5C, an image 520 is shown representing a set of Fourier coefficients resulting from the 2-D Fourier transform process applied to the wavelet coefficients generated from ultrasound image 500 of FIG. 5A (e.g., shown in vertical artifact detail image 514), via the wavelet decomposition process described above. In image 520, the set of Fourier coefficients indicate a presence of a first artifact 522 and a second artifact 524. While vertical artifact data in vertical artifact detail image 514 corresponds to vertical stripes in ultrasound image 500, first artifact 522 and second artifact 524 appear as horizontal lines as a result of the 2-D Fourier transform process.

Returning to FIG. 4, at 408, method 400 includes removing artifact data (e.g., first artifact 522 and second artifact 524) from the Fourier coefficients using a notch filter. The notch filter may remove a narrow band of frequencies from image 520, leaving frequencies outside the narrow band unchanged. Referring briefly to FIG. 5D, an image 530 shows a representation of a notch filter 532. Notch filter 532 may be applied to the Fourier coefficients (resulting from the 2-D Fourier transform) shown in FIG. 5C, which may remove first artifact 522 and a second artifact 524. FIG. 5E shows a notch filtered FFT image 540, with first artifact 522 and second artifact 524 removed.

Returning to FIG. 4, at 410, method 400 includes performing an inverse discrete Fourier transform (IDFT) on the filtered Fourier coefficients generated using the notch filter (e.g., notch filter 532), to generate a set of updated or regenerated wavelet coefficients corresponding to the first portion of the initial wavelet coefficients described above. The updated or regenerated wavelet coefficients may be the same as the first portion of the initial wavelet coefficients, but with the image artifact data substantially removed.

At 412, method 400 includes performing N level wavelet reconstruction on the wavelet coefficients of the ultrasound image, which include both the set of updated or regenerated wavelet coefficients corresponding to the first portion of the initial wavelet coefficients generated by the IDFT process described above, and the second portion of wavelet coefficients including no image artifact data (e.g., to which the 2-D Fourier transforms was not applied). In other words, the first portion of wavelet coefficients includes the artifact data. To remove the artifact data, the first portion of wavelet coefficients are transformed via the 2-D Fourier transform, and the artifact data is removed from the Fourier coefficients generated by the 2-D Fourier transform. The generated Fourier coefficients (with the artifacts removed) representing the first portion are then transformed back into wavelet coefficients via the IDFT process. The N level wavelet reconstruction is then performed on both the first and second portions of the wavelet coefficients to reconstruct an artifact-removed image. The artifact-removed image has the vertical artifacts (e.g., indicated by first artifact 522 and second artifact 524 of FIG. 5C) removed. As an example, FIG. 5F shows an artifact-removed image 550 resulting from wavelet reconstruction. When FIG. 5F is compared to the input ultrasound image 500 of FIG. 5A, it can be observed that the vertical stripes 502 of ultrasound image 500 have been reduced.

At 412, method 400 includes displaying the artifact-removed image on a display device (e.g., display device 118 or display device 234). Method 400 ends.

Thus, systems and methods are provided for removing visual artifacts such as stripes from medical images, even when the artifacts are of different sizes or at different scales and/or do not have well-defined boundaries. In accordance with the methods described herein, a wavelet decomposition is performed on each of N levels as described above; 2-D Fourier transforms are applied to selected wavelet coefficients resulting from the wavelet decomposition to generate Fourier coefficients, where the selected wavelet coefficients include image artifact data; some or all of the image artifact data is removed from the Fourier coefficients using a customized filter; the updated Fourier coefficients are converted back to the selected wavelet coefficients via an inverse 2-D Fourier transform; and then a wavelet reconstruction is performed on both the selected wavelet coefficients and the wavelet coefficients from the initial wavelet decomposition that were not selected for the 20D Fourier transforms, to reconstruct an output image. The output image may be a high-quality image with the artifacts partially or fully removed. By adjusting the type of wavelets selected, the type of 2-D transforms applied, and a design of the notch filter, various settings of a medical imaging system or image processing system may be accommodated, such as different decimation factors, aperture settings, types of ultrasound probes, reconstruction planes and reconstruction methods. As a result, the systems and methods may support acquisitions at a higher volume rate than other artifact-removal techniques, allowing for real-time artifact removal. Overall, a quality of images reconstructed by an imaging system may be increased, without affecting a workflow of an operator of the imaging system or slowing an acquisition rate.

The technical effect of applying a sequence of transforms including a wavelet transform, a 2-D Fourier transform, and a notch filter to ultrasound image data to remove visual artifacts from ultrasound images is that a quality of the ultrasound images is increased.

The disclosure also provides support for a method for an image processing system, comprising: receiving a medical image, performing a wavelet decomposition on image data of the medical image to generate a set of wavelet coefficients, identifying a first portion of the wavelet coefficients including image artifact data, and a second portion of the wavelet coefficients not including the image artifact data, performing one or more 2-D Fourier transforms on the first portion of the wavelet coefficients to generate Fourier coefficients, the Fourier coefficients including the image artifact data, removing the image artifact data from the Fourier coefficients generated from the one or more 2-D Fourier transforms, using a filter, performing an inverse 2-D Fourier transform on the filtered Fourier coefficients to generate updated wavelet coefficients corresponding to the first portion, reconstructing an artifact-removed image from the updated wavelet coefficients corresponding to the first portion of the wavelet coefficients and the second portion of the wavelet coefficients, and displaying the reconstructed, artifact-removed image on a display device of the image processing system. In a first example of the method, performing the wavelet decomposition on the image data further comprises: selecting a wavelet basis function for each level of N levels of the wavelet decomposition, each level corresponding to a different scale of the medical image, each wavelet basis function based on an initial wavelet basis function selected at a first level, for each level of the N levels, performing the wavelet decomposition, where a result of the wavelet decomposition includes an approximation image, a horizontal artifact detail image, a vertical artifact detail image, and a diagonal artifact detail image. In a second example of the method, optionally including the first example, the image data is used as in input to the wavelet decomposition performed at the first level. In a third example of the method, optionally including one or both of the first and second examples, the approximation image from a level of the N levels is used as an input into a wavelet decomposition performed at a subsequent level, and the image data is not used as an input into the wavelet decomposition. In a fourth example of the method, optionally including one or more or each of the first through third examples, selecting the initial wavelet basis function for the first level further comprises selecting the initial wavelet basis function based on a nature of an artifact in the image data. In a fifth example of the method, optionally including one or more or each of the first through fourth examples, performing the wavelet decomposition further comprises performing the wavelet decomposition to remove artifacts of more than one orientation. In a sixth example of the method, optionally including one or more or each of the first through fifth examples, performing the one or more 2-D Fourier transforms on the wavelet coefficients further comprises performing the one or more 2-D Fourier transforms on the first portion of the wavelet coefficients, and not performing the one or more 2-D Fourier transforms on the second portion of the wavelet coefficients. In a seventh example of the method, optionally including one or more or each of the first through sixth examples, the filter is a notch filter. In a eighth example of the method, optionally including one or more or each of the first through seventh examples, the method is applied to the medical image “on-the-fly” at a time of acquisition, and the artifact-removed image is displayed on the display device in real time. In a ninth example of the method, optionally including one or more or each of the first through eighth examples, the medical image is generated at a first time of acquisition and stored in the image processing system, and the method is applied to the medical image at a second, later time to remove the image artifact data prior to viewing the artifact-removed image on the display device. In a tenth example of the method, optionally including one or more or each of the first through ninth examples, the medical image is an ultrasound image obtained by any beamforming method such as Retrospective Transmit Beamforming (RTB), Synthetic Transmit Beamforming (STB), or a different beamforming method. In a eleventh example of the method, optionally including one or more or each of the first through tenth examples, the ultrasound image is one of a B-mode ultrasound image, a color or spectral Doppler ultrasound image, and an elastography image.

The disclosure also provides support for an image processing system, comprising: a processor, a non-transitory memory storing instructions that when executed, cause the processor to: perform a wavelet decomposition on image data of a medical image to generate a set of wavelet coefficients, identify a first portion of the wavelet coefficients including image artifacts, and a second portion of the wavelet coefficients not including the image artifacts, perform one or more 2-D Fourier transforms on the first portion of the wavelet coefficients to generate Fourier coefficients, the Fourier coefficients including the image artifacts, remove the image artifacts from the Fourier coefficients generated from the one or more 2-D Fourier transforms, using a filter, perform an inverse 2-D Fourier transform on the filtered Fourier coefficients to generate updated wavelet coefficients corresponding to the first portion, reconstruct an artifact-removed image from the updated wavelet coefficients corresponding to the first portion of the wavelet coefficients and the second portion of the wavelet coefficients, and display the reconstructed, artifact-removed image on a display device of the image processing system. In a first example of the system, performing the wavelet decomposition on the image data further comprises: based on a nature of the image artifacts, selecting an initial wavelet basis function for a first level of N levels of the wavelet decomposition, each level corresponding to a different scale of the medical image, determining additional wavelet basis functions for each additional level of the N levels based on the initial wavelet basis function, for a first level of the N levels, performing the wavelet decomposition on the image data using the initial wavelet basis function to generate an approximation image, a horizontal detail image, a vertical detail image, and a diagonal detail image, and for each subsequent level of the N levels, performing the wavelet decomposition on the approximation image from a previous level, using a wavelet basis function of the additional wavelet basis functions. In a second example of the system, optionally including the first example, for each subsequent level of the N levels, the image data is not an input into the wavelet decomposition. In a third example of the system, optionally including one or both of the first and second examples, performing the one or more 2-D Fourier transforms on the wavelet coefficients further comprises performing the one or more 2-D Fourier transforms on the first portion of the wavelet coefficients, and not performing the one or more 2-D Fourier transforms on the second portion of the wavelet coefficients. In a fourth example of the system, optionally including one or more or each of the first through third examples, the first portion of wavelet coefficients on which the 2-D Fourier transform is performed includes artifact data of one orientation at each of the N levels. In a fifth example of the system, optionally including one or more or each of the first through fourth examples, the first portion of wavelet coefficients on which the 2-D Fourier transform is performed includes artifact data of more than one orientation at each of the N levels.

The disclosure also provides support for a method for an ultrasound system, comprising: acquiring an ultrasound image via a probe of the ultrasound system during a scan of a subject, performing a wavelet decomposition of the ultrasound image to generate a set of wavelet coefficients, performing one or more 2-D Fourier transforms on selected wavelet coefficients of the set of wavelet coefficients to generate a set of Fourier coefficients, the selected wavelet coefficients including image artifact data, removing the image artifact data from the set of Fourier coefficients using a notch filter, regenerating the selected wavelet coefficients from the set of Fourier coefficients with the image artifact data removed, using inverse 2-D Fourier transforms, reconstructing an artifact-removed image using the regenerated wavelet coefficients, and displaying the reconstructed, artifact-removed image on a display device of the ultrasound system during the scan.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for an image processing system, comprising:
receiving a medical image;
performing a wavelet decomposition on image data of the medical image to generate a set of wavelet coefficients;
identifying a first portion of the wavelet coefficients including image artifact data, and a second portion of the wavelet coefficients not including the image artifact data;
performing one or more 2-D Fourier transforms on the first portion of the wavelet coefficients to generate Fourier coefficients, the Fourier coefficients including the image artifact data;
removing the image artifact data from the Fourier coefficients generated from the one or more 2-D Fourier transforms, using a filter;
performing an inverse 2-D Fourier transform on the filtered Fourier coefficients to generate updated wavelet coefficients corresponding to the first portion;
reconstructing an artifact-removed image from the updated wavelet coefficients corresponding to the first portion of the wavelet coefficients and the second portion of the wavelet coefficients; and
displaying the reconstructed, artifact-removed image on a display device of the image processing system.

2. The method of claim 1, wherein performing the wavelet decomposition on the image data further comprises:
selecting a wavelet basis function for each level of N levels of the wavelet decomposition, each level corresponding to a different scale of the medical image, each wavelet basis function based on an initial wavelet basis function selected at a first level;
for each level of the N levels, performing the wavelet decomposition, where a result of the wavelet decomposition includes an approximation image, a horizontal artifact detail image, a vertical artifact detail image, and a diagonal artifact detail image.

3. The method of claim 2, wherein the image data is used as in input to the wavelet decomposition performed at the first level.

4. The method of claim 2, wherein the approximation image from a level of the N levels is used as an input into a wavelet decomposition performed at a subsequent level, and the image data is not used as an input into the wavelet decomposition.

5. The method of claim 2, wherein selecting the initial wavelet basis function for the first level further comprises selecting the initial wavelet basis function based on a nature of an artifact in the image data.

6. The method of claim 5, wherein performing the wavelet decomposition further comprises performing the wavelet decomposition to remove artifacts of more than one orientation.

7. The method of claim 1, wherein performing the one or more 2-D Fourier transforms on the wavelet coefficients further comprises performing the one or more 2-D Fourier transforms on the first portion of the wavelet coefficients, and not performing the one or more 2-D Fourier transforms on the second portion of the wavelet coefficients.

8. The method of claim 1, wherein the filter is a notch filter.

9. The method of claim 1, wherein the method is applied to the medical image “on-the-fly” at a time of acquisition, and the artifact-removed image is displayed on the display device in real time.

10. The method of claim 1, wherein the medical image is generated at a first time of acquisition and stored in the image processing system, and the method is applied to the medical image at a second, later time to remove the image artifact data prior to viewing the artifact-removed image on the display device.

11. The method of claim 1, wherein the medical image is an ultrasound image obtained by any beamforming method such as Retrospective Transmit Beamforming (RTB), Synthetic Transmit Beamforming (STB), or a different beamforming method.

12. The method of claim 11, wherein the ultrasound image is one of a B-mode ultrasound image, a color Doppler or spectral Doppler ultrasound image, and an elastography image.

13. An image processing system, comprising:
a processor;
a non-transitory memory storing instructions that when executed, cause the processor to:
perform a wavelet decomposition on image data of a medical image to generate a set of wavelet coefficients;
identify a first portion of the wavelet coefficients including image artifacts, and a second portion of the wavelet coefficients not including the image artifacts;
perform one or more 2-D Fourier transforms on the first portion of the wavelet coefficients to generate Fourier coefficients, the Fourier coefficients including the image artifacts;
remove the image artifacts from the Fourier coefficients generated from the one or more 2-D Fourier transforms, using a filter;
perform an inverse 2-D Fourier transform on the filtered Fourier coefficients to generate updated wavelet coefficients corresponding to the first portion;
reconstruct an artifact-removed image from the updated wavelet coefficients corresponding to the first portion of the wavelet coefficients and the second portion of the wavelet coefficients; and
display the reconstructed, artifact-removed image on a display device of the image processing system.

14. The image processing system of claim 13, wherein performing the wavelet decomposition on the image data further comprises:
based on a nature of the image artifacts, selecting an initial wavelet basis function for a first level of N levels of the wavelet decomposition, each level corresponding to a different scale of the medical image;
determining additional wavelet basis functions for each additional level of the N levels based on the initial wavelet basis function;
for a first level of the N levels, performing the wavelet decomposition on the image data using the initial wavelet basis function to generate an approximation image, a horizontal detail image, a vertical detail image, and a diagonal detail image; and
for each subsequent level of the N levels, performing the wavelet decomposition on the approximation image from a previous level, using a wavelet basis function of the additional wavelet basis functions.

15. The image processing system of claim 14, wherein for each subsequent level of the N levels, the image data is not an input into the wavelet decomposition.

16. The image processing system of claim 13, wherein performing the one or more 2-D Fourier transforms on the wavelet coefficients further comprises performing the one or more 2-D Fourier transforms on the first portion of the wavelet coefficients, and not performing the one or more 2-D Fourier transforms on the second portion of the wavelet coefficients.

17. The image processing system of claim 16, wherein the first portion of wavelet coefficients on which the 2-D Fourier transform is performed includes artifact data of one orientation at each of the N levels.

18. The image processing system of claim 16, wherein the first portion of wavelet coefficients on which the 2-D Fourier transform is performed includes artifact data of more than one orientation at each of the N levels.

19. A method for an ultrasound system, comprising:
acquiring an ultrasound image via a probe of the ultrasound system during a scan of a subject;
performing a wavelet decomposition of the ultrasound image to generate a set of wavelet coefficients;
performing one or more 2-D Fourier transforms on selected wavelet coefficients of the set of wavelet coefficients to generate a set of Fourier coefficients, the selected wavelet coefficients including image artifact data;
removing the image artifact data from the set of Fourier coefficients using a notch filter;
regenerating the selected wavelet coefficients from the set of Fourier coefficients with the image artifact data removed, using inverse 2-D Fourier transforms;
reconstructing an artifact-removed image using the regenerated wavelet coefficients; and
displaying the reconstructed, artifact-removed image on a display device of the ultrasound system during the scan.

20. The method of claim 19, wherein the one or more 2-D Fourier transforms are not performed on the entire set of wavelet coefficients.
